(19) United States
(12) Patent Application Publication
(10) Pub. No.: US 2015/0367086 A1
(43) Pub. Date: Dec. 24, 2015

(54) TEMPERATURE CONTROLLING CASING FOR INJECTION TUBE

(71) Applicant: VYGON (SOCIÉTÉ ANONYME), Ecouen (FR)

(72) Inventors: Stian Haug LØVLIEN, Hamar (NO); Christer KITDAL, Hamar (NO)

(21) Appl. No.: 14/763,246

(22) PCT Filed: Jan. 31, 2014
(86) PCT No.: PCT/EP2014/051877

§ 371 Date: Jul. 24, 2015

(30) Foreign Application Priority Data
Jan. 31, 2013 (EP) 13153400.0

(51) Int. Cl. A61M 5/44; A61M 39/08

(52) U.S. Cl. CPC A61M 5/44; A61M 39/08; A61M 2205/3368; A61M 2205/36

(57) ABSTRACT

The invention concerns a temperature controlling device for controlling the temperature of transfer fluid intended to be injected into a living object. The device comprises a temperature controlling, hollow casing the casing being configured to partly enclose the longitudinal length of an injection tube. The hollow casing is further provided with a longitudinal casing slit extending along the casing's longitudinal direction for releasable arrangement of the injection tube there through. The hollow casing further comprises an inner casing section displaying a longitudinal through-going hollow casing opening and an outer casing section at least partly surrounding the inner casing, wherein the configuration of the inner casing and the outer casing ensures that the longitudinally directed heat conductivity of the inner casing is higher then the radially directed heat conductivity of the outer casing. The invention also concerns a method for the assembly of an injection tube into the inventive device.

### TECHNICAL FIELD

The present invention concerns a temperature controlling device for controlling the temperature of transfer fluid intended to be injected into a living object in accordance with the preamble of claim 1, and a method assembling such a temperature controlling device. In particular, the invention concerns a temperature controlling device for providing fluid into a living object for which a constant or near constant injection fluid temperature is of importance for the health and/or comfort of the living object.

The device is particularly suited for use in pre-hospital situations, for example accident scenes. However, it may also be applied for other medical purposes where transfer of fluid is pertinent.

### BACKGROUND TECHNIQUE

Transfer of temperate liquid in connection with critical circumstances such as accidents may be decisive for the topical humans or animals. If the liquid cools down below an expected value the transfer could cause larger damages than good (see e.g. E. Soreide, C. E. Smith, Scand J Trauma Resusc Emerg Med 2004; 12; 229-231). Such liquid cooling occurs when the temperate liquid is exposed to the cooler environment, and may take place both in the form of heat loss from the liquid container and heat loss during the actual transfer process from the container to the receiving patient. Stable liquid temperature within the container can easily be achieved by use of commercially available storage media such as thermal isolating containers. However, liquid transfer cooling is still considered a significant problem, in particular during pre-hospital situations such as at an accident scene.

In the field of medicine, it is known to use electric heating of resilient tube with temperature sensors. For example, US4'048'519 discloses a resilient tube of transparent plastic material having at least one helical shaped conductor and at least one temperature measuring resistance sensor in the form of a wire, where the conductor and the wire are twisted around the same axis. This solution allows satisfactory temperature and heating control. However, the solution requires the manufacturing of already assembled products in standardized or user customized lengths, causing high costs and limited flexibility and availability. Other devices of similar type may be found in US2002156451, EP1066844, WO9632080, DE2454349, EP0201985, EP102007056169 and U.S. Pat. No. 7,357,786.

### THE OBJECT OF THE INVENTION

Based on the state of the art, the object of the present invention is to provide an temperature controlling device that may be assembled easily and swiftly using injection tubes of non standardized dimensions such as tube length, and where the device allows a high degree of injection fluid temperature control during transfer into a living object such as a human being.

### SUMMARY OF THE INVENTION:

The above-identified object is achieved by a temperature controlling device as stated in claim 1, and injection device as stated in claim 14 and a method comprising the steps of claim 15. Further beneficial features are defined in the remaining dependent claims.

In particular, the present invention concerns a temperature controlling device for controlling the temperature of transfer fluid such as blood from an injection fluid source and into a living object. The device comprises a temperature controlling, hollow casing, where the casing is configured to partly enclose the longitudinal length of an injection tube suitable for transporting the injection fluid at least part of the distance from an injection fluid source to the living object, thereby providing thermal isolations to the tube during fluid transport. The interior diameter of the casing, i.e. the diameter of the hollow opening part, is preferably equal or larger than the exterior diameter of the injection tube. Furthermore, a longitudinal slit is provided along the casings' longitudinal direction for allowing releasable arrangement of the injection tube there through, preferably without requiring any interruption of the fluid transfer process. Note that the formulation “an injection tube” should hereinafter be interpreted as one tube or a bundle of more than one tube. This tube is preferably made of resilient material. The invention is further characterized in that the hollow casing comprises an inner casing section displaying a longitudinal through-going hollow casing opening and an outer casing section at least partly surrounding the inner casing, wherein the particular configuration of the inner casing section and the outer casing section, for example by the specific choice of manufacturing materials, ensures that the longitudinally directed heat conductivity of the inner casing section is higher then the radially directed heat conductivity of the outer casing section. In this way an enhanced temperature distribution as well as an increased thermal isolation is ensured for the transfer fluid during use. The choice of manufacturing material of the inner casing section may advantageously be such that the resulting heat conductivity in the longitudinal direction is at least 1 Wm⁻¹K⁻¹. An example of manufacturing material fulfilling this criterion is Thermally Conductive Elastomer (COPE) having a thermal conductivity of about 2.3 Wm⁻¹K⁻¹. Furthermore, the choice of manufacturing material of the outer casing section may advantageously be such that the resulting heat conductivity in the radial direction is less than 1 Wm⁻¹K⁻¹. An example fulfilling this criterion is some types of silicon rubbers.

In a preferred embodiment the interface between the inner casing section and the outer casing section displays at least one cavity, where at least one may extend from one longitudinal end of the casing to the other longitudinal end of the casing. More preferably at least one of cavity/channel is filled with a liquid or gas exhibiting high thermal insulating properties, for example air. Alternatively, at least one cavity/channel is under vacuum. These cavities/channels contribute to the desired high thermal insulation properties in the radial direction. Instead of having an inner casing section and an outer casing section it is also possible to apply an integrated hollow casing displaying a channel separated by spacers.

In another preferred embodiment the device also comprises a temperature sensor, more preferably two temperature sensors, for indicating the temperature in the injection fluid during use. This/these sensor(s) may be of type infrared thermometer and is/are advantageously arranged somewhere at the interface between the casing and the injection tube, for example within the inner casing section at one or both longitudinal ends of the device. Typically the temperature sensors form part of a prefabricated casing.

In another preferred embodiment the casing slit includes longitudinally directed surfaces forming a truncated V-shape into the hollow space of the casing which narrows down towards the hollow opening part of the casing. The entire casing may advantageously enclose only part of the outer circumference of the injection tube, for example between 50 and 95% of the circumference, more preferably 70 and 90%, typically around 80%. Outer circumference means hereinafter the effective circumference. Hence, if a bundle of more than one injection tubes is used, the circumference is the total circumference of all tubes after bundling.

In yet a preferred embodiment a coupling unit displaying a through-going hollow coupling unit opening is arranged on or near at least one, more preferably both, of the longitudinal ends of the casing, thereby allowing additional fixing of the injection tube inside the hollow opening part of the casing after assembly. Again, the configuration of the coupling unit(s) is/are configured to ensure swift and easy assembling, for example by introducing a coupling unit slit in the coupling unit which is fully aligned with the casing slit. However, the geometry of the casing slit and the coupling unit slit may vary in accordance with the purpose and requirement of the temperature controlling device. The coupling unit slit(s) may have an identical or similar shape as the above mentioned casing slit.

In yet a preferred embodiment at least one of the coupling unit(s) comprises a quick release coupler, or at least the bridging part of the coupler, for easy coupling of a power cable to any electric heating cables during assembly. The design of such a quick release coupler can be of any known type. The quick release coupler, or at least the bridging part of the coupler, may alternatively, or additionally, be arranged on at least one of the electric cables intended to be coupled to the electric heating cables.

In yet a preferred embodiment the casing is further provided with at least one cavity or track/channel extending longitudinally along the casing for arrangement of heating cables and/or communication wires therein. These cables should then be connected to a suitable electric power supply to ensure controllable heating of the injection tube, for example by the above mentioned power cable(s). The device may be pre-manufactured with at least one heating cable situated within at least one of the at least one channel for user controlled heating of at least part of the casing, including a grabbing part or a receiving part of at least one quick release coupler. The at least one channel for arrangement of heating cables extends preferably within the inner casing section.

In yet a preferred embodiment the interior axial opening walls of the casing, for example the interior axial opening walls of the inner casing section, is at least partly covered by one or more fluorescent materials, thereby facilitating the user ability to visually control the flow of fluid during transfer.

Furthermore, the casing may be produced by at least one material emitting light with distinct fluid temperature depending light properties such as light intensity and/or light frequency.

The invention concerns as well an injection device comprising an injection tube for flow of injection fluid, a temperature controlling device in accordance with any of the features disclosed above and a power supply cable connected to at least one longitudinal end of the temperature controlling device for supply of power to at least one of a communication wire and a heating cable situated within the temperature controlling device.

The invention also concerns method for assembling a temperature controlling casing with a hollow opening part onto an injection tube in order to achieve a temperature controlling device in accordance with any of the features mentioned above. The method comprises the following steps:
enclosing at least partly one longitudinal end of the casing around a part of the injection tube, forming an assembled end of the temperature controlling device, and
applying an external force on one or both of the casing and the injection tube to force the injection tube into the hollow part of the casing.

Said the external force may be applied manually, for example by by performing a sliding movement on the injection tube along the casing slit and any coupling unit slit(s).

In the following description, numerous specific details are introduced to provide a thorough understanding of embodiments of the claimed apparatus. One skilled in the relevant art, however, will recognize that these embodiments can be practiced without one or more of the specific details, or with other components, systems, etc. In other instances, well-known structures or operations are not shown, or are not described in detail, to avoid obscuring aspects of the disclosed embodiments.

### SHORT SUMMARY OF THE DRAWINGS

Preferred embodiments of the present invention will now be described with reference to the attached drawings, in which:

FIG. 1 is a perspective illustration of one end of an injection device in accordance with a first embodiment of the invention showing a temperature controlling, hollow casing partly surrounding a resilient injection tube and a corresponding coupling unit,

FIG. 2 is a schematic illustration of the radial cross section of the hollow casing according to FIG. 1 viewed along the longitudinal direction of the casing,

FIG. 3 is a schematic illustration of a longitudinal cross section of the hollow casing according to FIG. 1 viewed along the radial direction of the casing,

FIG. 4 is a perspective illustration of the end of the injection device according to FIG. 1 showing a quick release coupler in locked position and a power cable assembled to the coupling unit,

FIG. 5 is a perspective illustration of the end of the injection device according to FIG. 4 showing the power cable in a connected state and the quick release coupler in unlocked position,

FIG. 6 is a perspective illustration of the end of the injection device according to FIGS. 4 and 5 showing the power cable in a disconnected state and

FIG. 7 is a perspective illustration of an end of the temperature controlling device according to the first embodiment of the invention, showing details of the hollow casing including the casing slit and the cavities/channels within the casing.

FIG. 8 is a perspective illustration of an injection device according to a second embodiment of the invention showing a split drawing of the injection device comprising a temperature controlling device having hollow casing sections that partly surround a resilient injection tube,

FIG. 9 is a top view of the injection device of FIG. 8,

FIG. 10 is a radial cross sectional view of an inner casing section,

FIG. 11 is a radial cross sectional view of an outer casing section and

FIG. 12 is a front view drawing of a control unit.

### DETAILED DESCRIPTION OF THE INVENTION

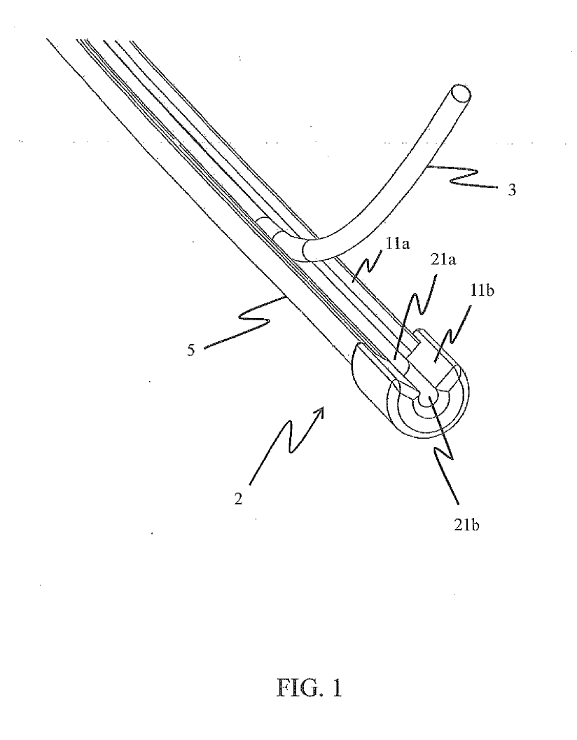

The injection device 1 shown in FIG. 1 presents a first embodiment of the invention and comprises an injection tube 3 and a temperature controlling device 2, where the temperature controlling device 2 further comprises an temperature isolating casing 5 and a coupling unit 8a,8b arranged at one or both ends of the casing 5. Both the casing 5 and the coupling unit 8a,8b are provided with a hollow opening 21a,21b and slits 11a,11b adapted to receive the resilient injection tube 3. The casing slit 1 la and the coupling unit slit 11b, as well as the corresponding openings 21a,21b, extend all along the longitudinal length of the temperature controlling device 2. Both the injection tube 3 and the isolating casing 5 are advantageously made of resilient materials such as various forms of rubber. As is apparent from the figure the injection tube 3 may be guided into the corresponding temperature controlling device 2 by initially pushing the tube 3 through the coupling unit slit 11b at one of the two ends of the temperature controlling device 2. This pushing act is then continued all along the longitudinal length of the temperature controlling device 2 until the opposite end is reached, for example by using one or more fingers. The purpose of the cylindrically formed coupling units 8a,8b situated at the two ends of the casing 5 is primarily to better fasten the injection tube 3 to the temperature controlling device 2 and to provide suitable interfaces for external power supplies 12. In FIG. 1 only the coupling unit 8b is shown, that is without such interfaces. The coupling units 8a,8b may be manufactured as separate units and then arranged onto the casing ends, or be integral parts of the casing 5, or a combination thereof. The hollow opening 21b and the slit 11b of the coupling units 8a,8b should advantageously be aligned with the corresponding hollow opening 21a and slit 11a of the casing 5. To ease the understanding of the assembling method the injection device 1 is shown in FIG. 1 in a not fully completed state. Since the stability of the assembled injection tube 3 is in this particular embodiment obtained by ensuring a tight fit with the coupling units 8a,8b at the casing ends the internal diameter of the hollow opening part 21a of the casing 5 may be somewhat larger than external diameter of the injection tube 3. However, the internal opening diameter of the coupling units 8a,8b should advantageously be equal (or close to equal) to the tube diameter. Of course, other locking systems between the tube 3 and the casing 5 may be envisaged such as mechanical lock(s), burr fasteners (Velcro®) or systems based on adhesives.

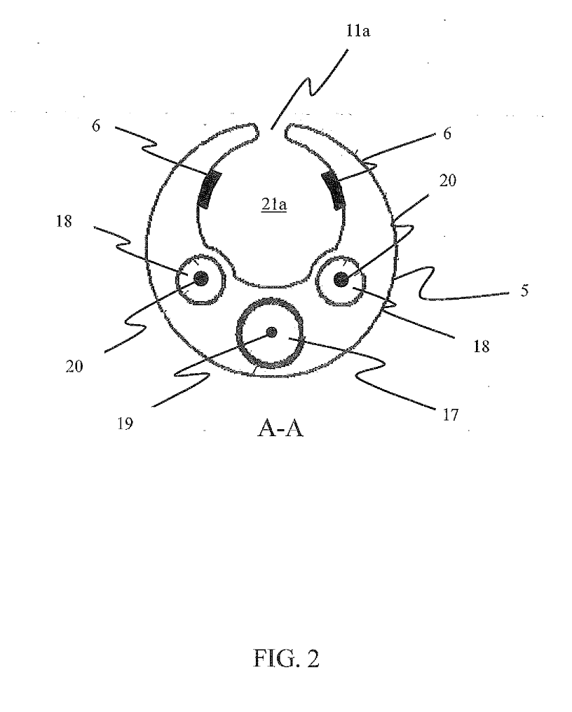

FIG. 2 shows a schematic illustration of the radial cross section of the casing 5 taken along line A-A in FIG. 1. The resilient properties of the casing slit 11a are apparent. In addition to the axial casing opening 21a and the casing split 11a, FIG. 2 also shows three channels 17,18 where the centred channel 17 and the two side channels 18 are intended to house one or more communication wires 19 and one or more heating cables 20, respectively. The longitudinal extending heating cable(s) 20 allows controllable heating of the fluid running through the injection tube 3 (such as IV-tube) during use. Further, the similarly arranged communication wires 19 allows transmittal of other electronic signals along the fluid injection path in order to allow other applications which requires receipt and/or transmittal of such signals. Of course, the purposes of the different channels 17,18 may be interchanged as convenient. For example, all the channels 17,18 may contain heating cables 20 only. Furthermore, two temperature sensors 6 are indicated coupled to the interior walls of the casing 5. These sensors are typically of type infrared thermometers and are (as FIG. 2 illustrates) preferably located at the interface between the interior (axial opening) wall of the casing 5 and the exterior wall of the injection tube 3 when the injection device 1 is assembled. Alternatively, or in addition, the sensors 6 may be arranged at the interior walls of one or both of the coupling units 8 and/or on the end 13 of any cable(s) 12 intended to be connected to the temperature controlling device 2.

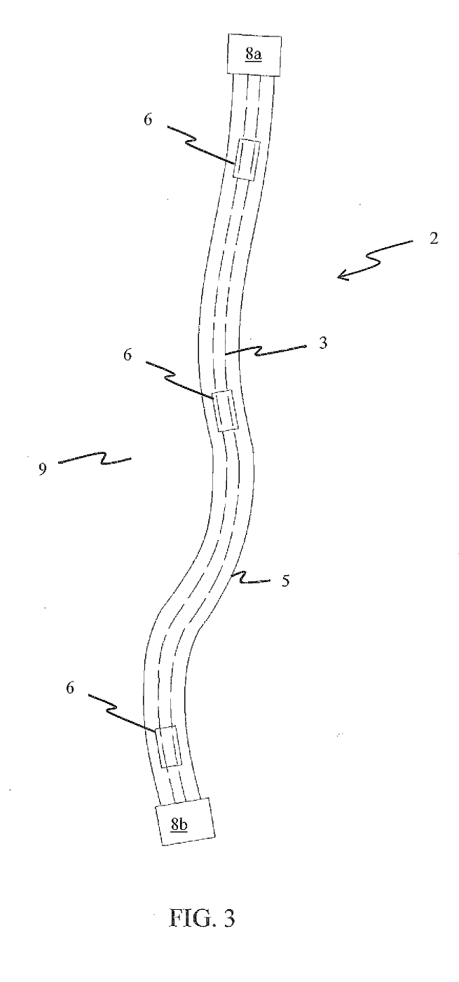

FIG. 3 shows a schematic illustration of the longitudinal cross section of the temperature controlling device 2 showing the casing 5, one of the coupling units 8a and exemplary positions of two temperature sensors 6 situated on the interior wall of the casing 5. The dotted lines along the casing indicate the position of the assembled injection tube 3.

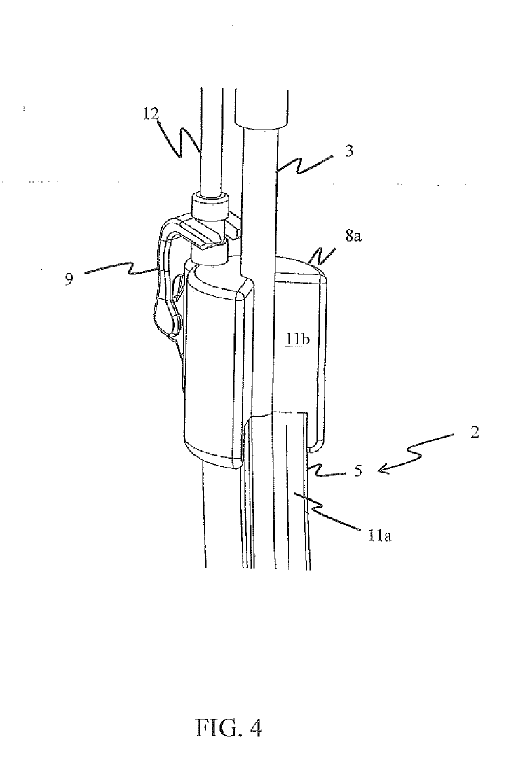

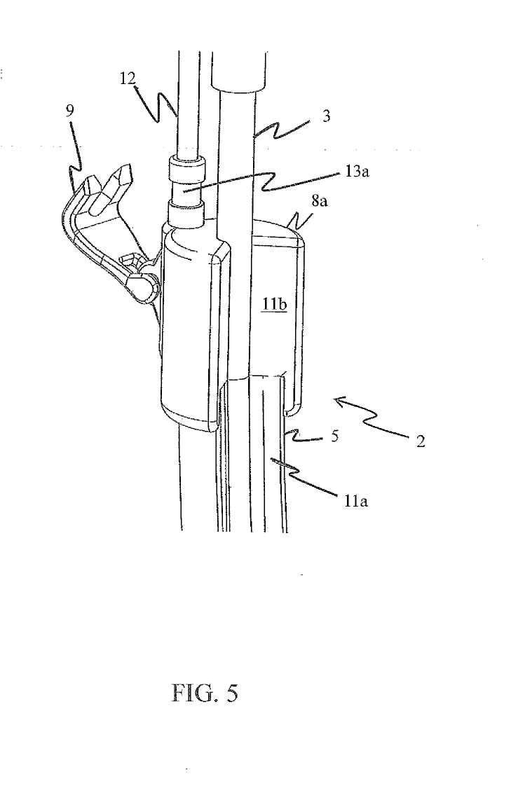

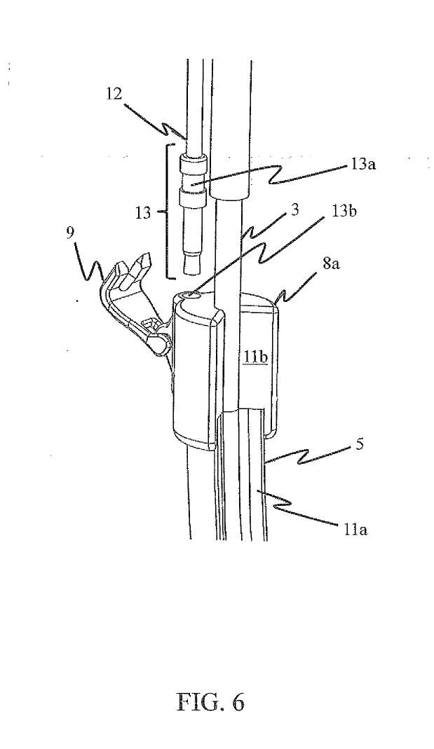

Further details of the power cable receiving coupling unit 8a situated at an end of the temperature controlling device 2 is illustrated in FIG. 4. The injection tube 3 is (at least at this end) completely inserted into the openings 21a,21b of the casing 5 and its corresponding coupling unit 8a, respectively. Furthermore, the power supply cable 12 is shown connected to one of the axial ends of the coupling unit 8 to allow supply of electrical power to for example the resistive heating cable 20 arranged parallel to the injection tube 3, thereby ensuring controllable heating of the injection fluid. The connection between the end 13 (see FIG. 6) of the power supply cable 12 and the corresponding coupling unit 8a is stabilized using a quick release coupler 9 of known type, in FIG. 4 shown as a pivoting fork grabbing the cable end 13 in a suitable quick release recess 13a. The pivoting property of the quick release coupler 9 is also illustrated in FIGS. 5 and 6 showing the situation as in FIG. 4 with the quick release coupler 9 in unlocked position. In FIGS. 5 and 6 the end 13 of the power supply cable 12 is illustrated in a connected and disconnected state, respectively. Hence, after inserting the injection tube 3 into the casing 5 and the coupling units 8a,8b the connection of any heating system(s) 18,20 along the injection tube 3 is easily achieved by connecting the end 13 of the power supply cable 12 into the appropriate socket 13b in one (or alternatively both) of the coupling units 8a, followed by pushing the fork of the quick release coupler 9 into a locked position. The opposite procedure is followed during disassembly of the temperature controlling device 1. Of course, the bridging part of the quick release coupler 9 may also be arranged on the power supply cable 12, with the quick release recess 13a being situated on the corresponding coupling unit 8a,8b.

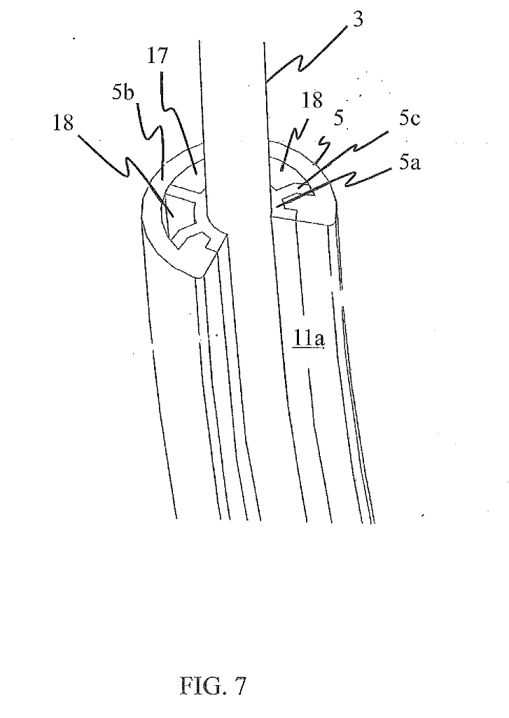

FIG. 7 shows further details of the interior of the casing 5 after removal of the coupling unit 8a,8b for one embodiment In this particular embodiment the radial innards is composed of longitudinally extending inner and outer casing cylinders 5a,5b, separated by longitudinally extending casing spacers 5c, hence forming the above mentioned casing channels 17,18. In addition to enable housing of cables and/or wires 19,20 an open structure between the outer and inner casing cylinders 5b,5a gives a desirable decrease in the total weight of the device 1.

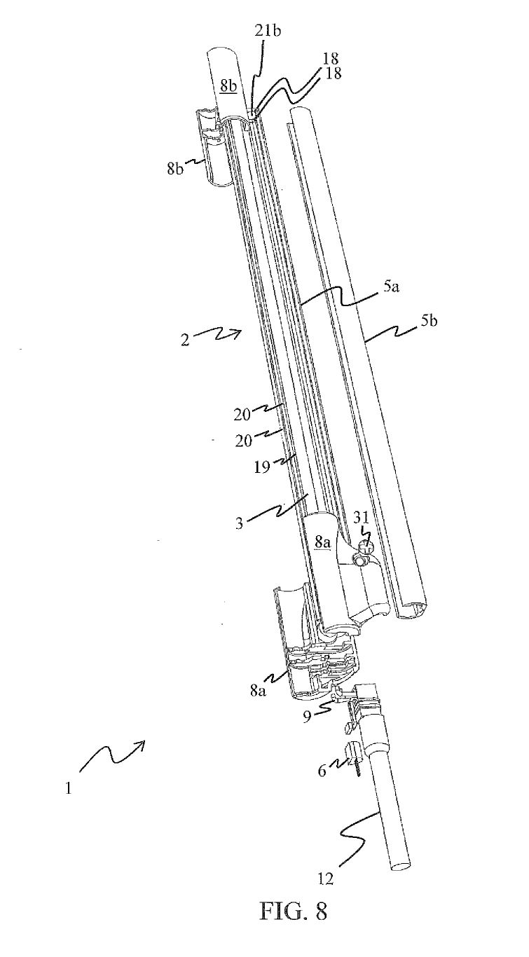

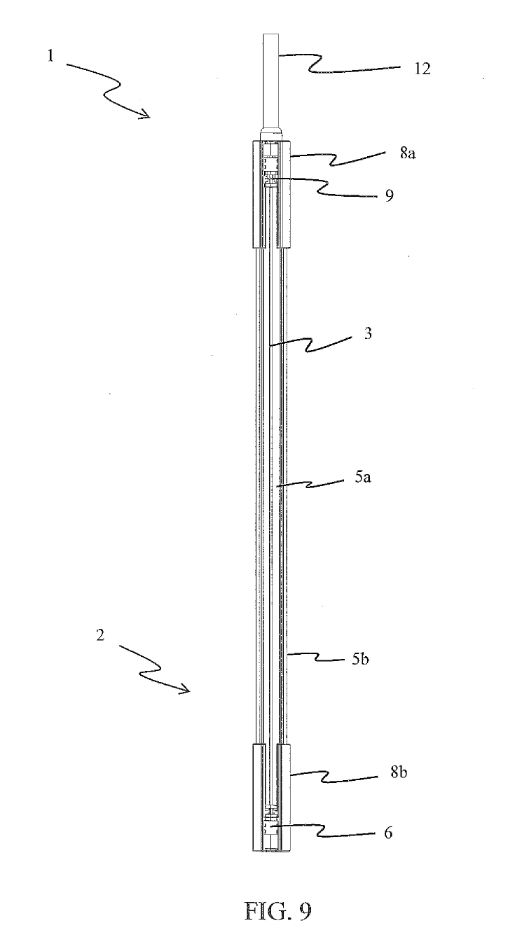

FIGS. 8-11 show a second embodiment of the invention where the temperature isolating casing 5 comprises two separate parts, an outer casing section 5b and an inner casing section 5a. As is apparent from the split drawing (FIG. 8) and the top view (FIG. 9) the outer casing sections 5b is arranged along, and in direct contact with, the inner casing section 5a, while the inner casing section 5a is arranged along, and in direct contact with, the injection tube 3. Furthermore, the inner casing section 5a is made of materials exhibiting high heat conducting properties, for example Thermally Conductive Copolyester Elastomer (COPE), to ensure high degree of uniform heating along the injection tube 3, and the outer casing section 5b is made of materials exhibiting high heat insulating properties, for example medical grade silicone, to ensure high degree of thermal insulation of the injection tube 3/fluid. FIG. 8 further shows the power supply cable 12 comprising a temperature sensor 6 and the bridging part of a quick release coupler 9 situated on or near the end 13 of the cable 12. The coupling units 8a,8b are shown as two longitudinally extending parts being assembled by a suitable fixating screw 31. The controlling device 2 in FIG. 8 is also illustrated with a longitudinal extending communication wire 19 (for example transmitting signals to temperature sensors 6), two longitudinally extending heating wires/cables 20 and two heating wire channels 18 for accommodating the two heating wires 20. The communication wires 19 may be arranged in corresponding communication wire channels 17 as shown in FIG. 2. The assembled controlling device 2 including the connected power supply cable 12 is illustrated in the top view FIG. 9.

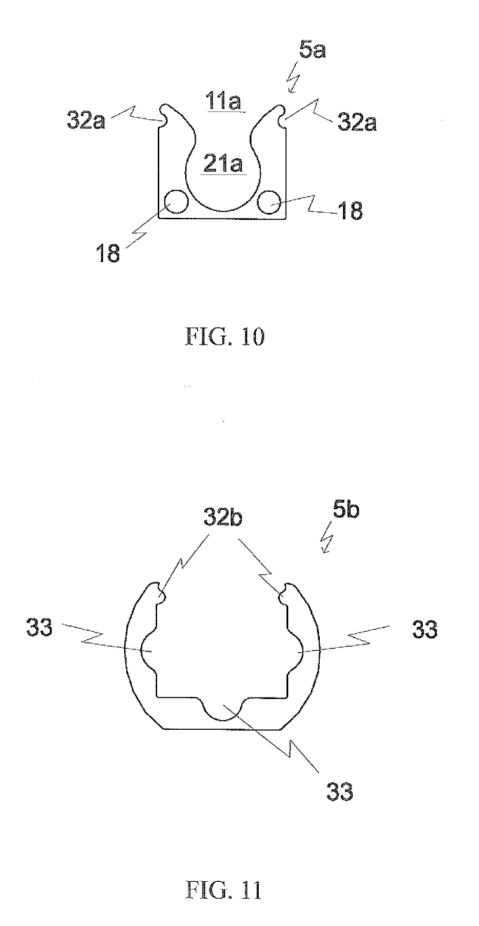

FIGS. 10 and 11 show cross sectional views of the inner and outer casing sections 5a,5b, respectively. The inner casing section 5a displays two heating wire channels 18 into which the heating wires 20 may be arranged (alternatively one channel 18 with heating wire 20 and one channel 18 with communication wire 19) and two grooves 32a for easy fastening of the outer casing section 5b. The inner casing section 5a further displays longitudinally extending opening 21a and casing slit 11a for insertion of the injection tube 3 therein. The outer casing section 5b displays three longitudinally extending isolation channels 33 for increasing the thermal insulation properties of the casing 5 in the radial direction. Said isolation channels 33 may also be used to accommodate wires such as some or all of the communication wires 19. Protrusions 32b for hooking into the corresponding grooves 32a are shown in the upper part of the outer casing section 5b.

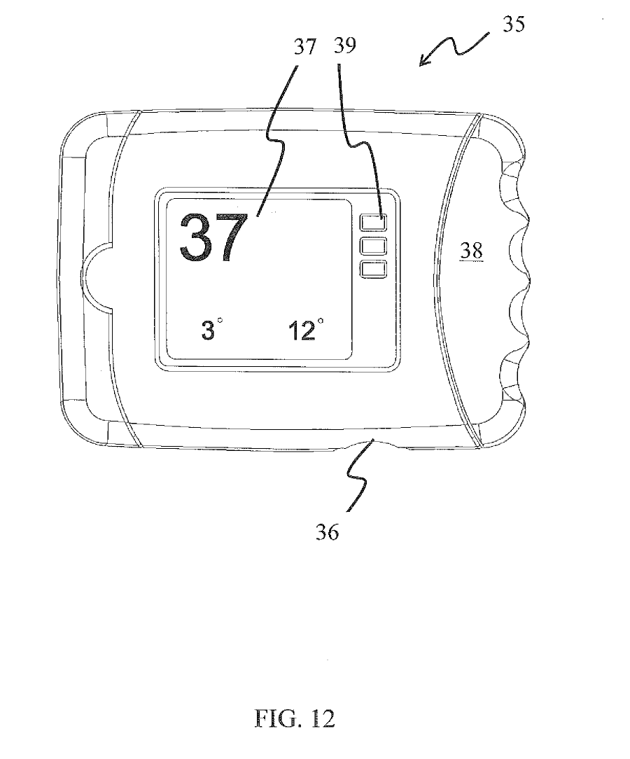

FIG. 12 shows an example of a control unit 35 that may be connected to one of the wires 19,20 and/or cables 12 in order to control and monitor the operation of the injection device 1. The control unit 35 comprises a connection socket 36 for connecting the control unit 35 to the temperature controlling device 2, a display panel 37 displaying relevant parameters during operation, buttons 39 for setting the control unit 35 and a battery compartment 38 for insertion of battery. In FIG. 12 the display panel shows as an example a fluid temperature of 37°, an outside temperature of 3° and a surface temperature of 12°.

In the preceding description, various aspects of the temperature controlling device and its corresponding method according to the invention have been described with reference to the illustrative embodiment. For purposes of explanation, specific numbers, systems and configurations were set forth in order to provide a thorough understanding of the device and its workings. However, this description is not intended to be construed in a limiting sense. Various modifications and variations of the illustrative embodiment, as well as other embodiments of the device, which are apparent to persons skilled in the art to which the disclosed subject matter pertains, are deemed to lie within the scope of the present invention.

1. Temperature controlling device for controlling the temperature of transfer fluid intended to be injected into a living object, comprising,
a temperature controlling, hollow casing, the casing being configured
to partly enclose the longitudinal length of an injection tube, wherein a longitudinal casing slit is provided along the casings' longitudinal direction for releasable arrangement of the injection tube there through, wherein the hollow casing further comprises
an inner casing section made of a material having heat conductivity in the longitudinal direction of at least 1 Wm⁻¹K⁻¹, displaying a longitudinal through-going hollow casing opening and
an outer casing section made of a material having heat conductivity in the radial direction of less than 1 Wm⁻¹K⁻¹, at least partly surrounding the inner casing, wherein the configuration of the inner casing and the outer casing ensures that the longitudinally directed heat conductivity of the inner casing is higher then the radially directed heat conductivity of the outer casing.

2. (canceled)

3. (canceled)

4. The temperature controlling device in accordance with claim 1, wherein the interface between the inner casing section and the outer casing section displays at least one cavity.

5. The temperature controlling device in accordance with claim 4, wherein at least one of the at least one cavity is a channel extending from one longitudinal end of the casing to the other longitudinal end of the casing.

6. The temperature controlling device in accordance with claim 1, wherein the device comprises a temperature sensor for sensing the temperature of the injection fluid during use.

7. The temperature controlling device in accordance with claim 1, wherein a coupling unit displaying an axial through-going hollow coupling unit opening is arranged on at least one of the longitudinal ends of the casing.

8. The temperature controlling device in accordance with claim 7, wherein at least one of the coupling unit(s) is provided with a coupling unit slit into and along the hollow coupling unit opening of the coupling unit(s), said slit being in longitudinal alignment with the casing slit.

9. The temperature controlling device in accordance with claim 1, wherein at least one channel extends longitudinally along the casing for arrangement of heating cables and communication wires therein.

10. The temperature controlling device in accordance with claim 9, wherein the device further comprises
at least one heating cable arranged within at least one of the at least one channel for user controlled heating of at least part of the casing and
at least a part of at least one quick release coupler for releasable coupling of the heating cable with a power supply cable during assembly.

11. The temperature controlling device in accordance with claim 9, wherein the at least one channel for arrangement of heating cables extends within the inner casing section.

12. The temperature controlling device in accordance with claim 1, wherein the interior walls of the inner casing section is at least partly covered by one or more fluorescent materials.

13. Temperature controlling device in accordance with claim 1, wherein at least part of the casing is produced of at least one material emitting light with distinct fluid temperature depending light properties.

14. An injection device comprising an injection tube, a temperature controlling device in accordance with any of the claim 1 and a power supply cable connected to at least one longitudinal end of the temperature controlling device for supply of power to at least one of a communication wire and a heating cable.

15. Method for assembling a temperature controlling, hollow casing onto an injection tube, comprising the following steps:
partly enclosing one longitudinal end of the temperature controlling device in accordance with claim 1 around part of the injection tube, thereby forming an assembled end of the injection device, and
applying an external force on one or both of the temperature controlling device and the injection tube to force the injection tube into the hollow part of the device.
